(19) United States
(12) Patent Application Publication
(10) Pub. No.: US 2005/0101632 A1
(43) Pub. Date: May 12, 2005

(54) Method for the treatment of inflammation

(76) Inventors: Lyne Gagnon, Quebec (CA); Pierre Laurin, Quebec (CA)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/415,793

(22) PCT Filed: Nov. 8, 2001
(86) PCT No.: PCT/IB01/02096

Related U.S. Application Data
(60) Provisional application No. 60/246,792, filed on Nov. 8, 2000.

(51) Int. Cl. A61K 31/47

(52) U.S. Cl. 514/313

(57) ABSTRACT

A method for treating, eliminating and preventing an inflammation of the skin or mucous membrane of a mammal, including a human, by administering to the skin or mucous membrane a composition containing dimethicone and a pharmaceutically acceptable carrier. The dimethicone composition is applied to the skin or mucous membrane in an amount effective to treat, eliminate or prevent the inflammation.

### FIELD OF THE INVENTION

The present invention relates to the fields of immunology and pharmacology and more particularly relates to a method for the treatment of inflammation.

### BACKGROUND OF THE INVENTION

Inflammation is a local response to cellular injury that initiates the elimination of noxious agents and damaged tissue. Inflammation is a complex process of physiological and immunological events characterized by capillary dilation, leukocyte infiltration, redness, heat, pain and swelling. Polymorphonuclear neutrophils (hereinafter, “PMNs”) and eosinophils are among the list of important mediators of inflammation and release growth factors, cytokines, prostaglandins, leukotrienes and proteases that exacerbate tissue damage. PMN-derived serine proteases such as elastase and cathepsin G are known pathogenic factors in inflammatory and degenerative diseases that include abnormal tissue catabolism.

Although the inflammatory response can be regulated by anti-inflammatory agents such as corticocosteroids, immunosuppressants, non steroidal anti-inflammatory drugs (NSAID), COX-2 inhibitors and protease inhibitors, many of these agents have significant side effects. Corticosteroids may induce Cushingoid features, skin thinning, increased susceptibility to infection and suppression of the hypothalamic-pituitary-adrenal axis. Immunosuppressants may induce hypertension and nephrotoxicity.

### BRIEF DESCRIPTION OF THE FIGURES

FIG. 1 is a bar graph of optical density versus dimethicone concentration showing the inhibitory effect of dimethicone on porcine elastase activity.

FIG. 2A a bar graph showing the effect of 0, 5.00% and 2.50% dimethicone on PMN phagocytosis in blood samples taken from eight human volunteers. FIG. 2B is a bar graph showing the effect of various concentrations of dimethicone on macrophage phagocytosis in blood samples taken from five human volunteers.

FIG. 3 is a graph showing the effect of various concentrations of dimethicone on PHA-stimulated PBML proliferation.

FIG. 4 is a bar graph of the increase of ear thickness following topical application of hydrocortisone (HC) or various concentrations of dimethicone (DM) showing the effect of hydrocortisone or dimethicone on a delayed-type hypersensitivity reaction in sensitized mice after challenge. The hypersensitivity reaction was an oxazolone-induced hypersensitivity reaction in oxazolone-sensitized mice. Treatment consisted of topical application of the drug twice daily for four days.

FIG. 5 is a bar graph of the increase of ear thickness following topical application of hydrocortisone (HC) or various. concentrations of dimethicone (DM) showing the effect of hydrocortisone or dimethicone on a delayed-type hypersensitivity reaction in sensitized mice after a second challenge. The hypersensitivity reaction was an oxazolone-induced hypersensitivity reaction in oxazolone-sensitized mice. Treatment consisted of topical application of the drug once daily for four days.

FIG. 6 is a bar graph of the increase of ear thickness following topical application of hydrocortisone (HC) or various concentrations of dimethicone (DM) showing the effect of hydrocortisone or dimethicone on a delayed-type hypersensitivity reaction in sensitized mice after a third challenge. The hypersensitivity reaction was an oxazolone-induced hypersensitivity reaction in oxazolone-sensitized mice. Treatment consisted of topical application of the drug once daily for four days.

FIG 7 is a graph of body weight of mice during topical application of hydrocortisone (HC) or various concentrations of dimethicone (DM) to oxazolone-sensitized mice after one, two or three challenges with oxazolone and 24, 48 and 72 hours after each challenge.

### DETAILED DESCRIPTION OF THE INVENTION

A method for treating an inflammation of skin or a mucous membrane of a mammal, including a human, is provided. In accordance with the method, a composition containing dimethicone and a pharmaceutically acceptable carrier is applied to the skin or mucous membrane in an amount effective to treat inflammation of the skin or mucous membrane. Treatment is useful for reducing, eliminating or preventing inflammation.

As used herein, the word “dimethicone” refers to a silicone oil having a mixture of fully-methylated linear siloxane polymers end-blocked with tiethylsilyloxy units, its analogs, salts, complexes and derivatives and simethicone (a mixture of dimethicone and silicone dioxide).

As used herein the term “dimethicone composition” refers to a composition containing dimethicone and one or more pharmaceutically acceptable carriers.

As used herein, the term “pharmaceutically acceptable” refers to a substance that does not interfere with the physiological effects of dimethicone and that is not toxic to mammals, including humans.

As used herein, the term “inflammation” refers to an inflammatory condition of the skin or of a mucous membrane.

As used herein, the phrases “effective amount” and “effective to treat” refer to the amount of a dimethicone composition that provides either subjective relief in the symptoms of inflammation as determined by the recipient or objective improvement in the symptoms of inflammation as determined by one skilled in the art.

Dimethicone has been used as an anti-flatulent (U.S. Pat. Nos. 5,248,505 and 5,908,636 and PCT Application No. W09703650A1); as a lubricant (U.S. Pat. Nos. 5,922,351, 5,980,944, 5,229,137; European Patent Application Nos. EP571217B1 and EP14253B1; Japanese Patent Application Nos. JP3203048A and JP45230329A; and PCT Application No. WO9610409A3); in hair products (U.S. Pat. Nos. 5,658,558, 5,437,809, 5,989,532 and 5,658,558; and Japanese Patent Application No. JP1305019A); and, in cosmetic products (U.S. Pat. Nos. 6,007,799, 5,882,666 and 5,208,013; and Japanese Patent Application No. JP1287008A; Disaprio et al. Int. J. Cosemt. Sci. 10:75, 1989). However, until the unexpected discovery described herein, the effectiveness of dimethicone in the treatment of an inflammation of the skin or mucous membranes was not known.

Inflammations suitable for treatment using the method describe herein include, but are not limited to, inflammation of the epidermis, dermis, eye, nasopharynx, gastrointestinal tract and urogenital tract. Such inflammations include, but are not limited to, acne; eczema; gingivitis; psoriasis; pruritus; decubitus ulcer; dermatitis such as allergic dermatitis, contact dermatitis, seborrheac dermatitis and mummular dermatitis; actinic keratosis; actinic lentigos; ichthyoses; ichtyposiformis; Darier maladay; palmoplantary keratodermies; leucoplasies; leucoplasiformis; lichen; blisters; collagen maladies; ultraviolet light damage; rosacea; melasma; comedons; polymorphs; conglobata; infection; sinusitis; dyshidrosis tonsillitis; buccal ulceration; gastrointestinal diseases such as gastric ulceration and inflammatory bowel disease such as colitis, ulcerative colitis, Crohn's disease, enteritis, and haemorrhoids; urogenital diseases; and, fungal, yeast, bacterial and viral dermatides. Application of the dimethicone composition also is effective in treating wounds, particularly wounds to the skin. Suitable wounds to be treated with the method described herein include, but not limited to, burns, bruises, scrapes, cuts and lacerations.

Most preferably for treatment of the skin, the dimethicone composition is administered topically. Alternatively, depending of course on the mucous membrane to be treated, the dimethicone composition is administered intranasally, intravaginally, intrarectally, or intraoccularly. Topical application includes transdermal or percutaneous delivery such as by application of a percutaneous occlusive patch.

The dimethicone composition to be administered in accordance with the method is formulated using dimethicone and one or more pharmaceutically acceptable carriers by methods known to those skilled in the art, such as, for example, DERMATOLOGICAL FORMULATIONS: PERCUTANEOUS ABSORPTION, Barry (ed.), Marcel Dekker Inc., 1983 and REMINGTON'S PHARMACEUTICAL SCIENCES). The composition may be in the form of, but is not limited to, a liquid, oil, emulsion, lotion, gel, cream, paste, ointment, foam, powder, aerosol, inhalant, patch or suppositories. The carrier may also be a diluent.

Pharmaceutically acceptable carriers for use in the present invention are known to those skilled in the art. Exemplary carriers can be found in scientific texts such as MERCK INDEX, Merck & Co., Rahway, N.J.; and REMINGTON'S PHARMACEUTICAL SCIENCES. Suitable carriers include, but are not limited to, dermophilics; emollients, bactericides, water; saline; and, buffers. Other pharmaceutically acceptable carriers include, but are not limited to, anti-oxidents; coloring agents; dying agents; perfumes; preservatives; tensioactive agents; emulsifying agents such as magnesium aluminum silicate, polyoxyethylene lauryl ether, polyoxyethylene monosteatate, polyoxyethylene sorbitan monolaurate, sorbitan monopalmitate, propylene glycol monostearate, sodium borate plus fatty acid, sodium lauryl sulfate, triethanolamine plus fatty acid, isopropyl myristate and waxes; preservatives such as cresol, propylparaben, methylparaben or sorbic acid; and, stabilizing or thickening agents such as carbomer, cetyl alcohol, glyceryl monostearate, methylcerlulose, spermaceti and stearyl alcohol.

Moreover, depending on the proposed use, the dimethicone composition may be administered in conjunction with other active agents including, but not limited to, cicatrizers, local anesthetics, keratolytics, antibiotics, anti-inflammatories, caprylic acid triglyceride, protease inhibitors, antifungals, antiseptics, chemotherapeutics, cytotoxics, nucleosides, immunosuppressors, anti-cancer agents, anti-infectious agents and antihistamines. Preferred anti-inflammatories include nonsteroidal anti-inflammatories, the protein alpha-1-antitrypsin and hyaluronic acid or analogues thereof. The active agents may be administered before, after, or simultaneously with the dimethicone in separate pharmaceutical formulations or combined in a dimethicone composition formulation and may be additive to or synergistic with dimethicone.

The dimethicone composition is formulated to deliver dinethicone directly onto the inflamed skin or mucous membrane. The dosage of dimethicone used depends on many factors known to those skilled in the art including, but not limited to, the inflammation being treated; the severity of the inflammation; the composition being used; the age, weight and clinical condition of the recipient; and, the experience and judgment of the individual administering the dimethicone. Typically, a single dosage contains from about 0.001 to 40% dimethicone, more preferably from about 0.01 to 25% dimetflicone and most preferably from about 0.1 to 15% dimethicone. The amount of dimethicone composition administered is preferably from about 0.01 to 100 ml, more preferably from about 0.05 to 7.5 ml and most preferably from about 0.1 to 5.0 ml.

The dimethicone composition may be conveniently presented in a unit dosage form prepared by conventional pharmaceutical techniques. A dosage of dimethicone can be administered one time or several times to the same recipient The dosage anount and the dosage schedule can be determined readily by those skilled in the art. Preferred dosage formulations are those containing a dose, sub-dose or fraction thereof of including, but not limited to, the formulations described herein. Further, it should be understood that in addition to the ingredients particularly mentioned herein, the present method can include other agents conventional in the art having regard to the type of formulation being used.

The carrier may also include a delayed release material, such as glyceryl monostearate or glyceryl disterearate alone or in combination with a wax. The composition may additionally contain conventional agents such as preservatives (including antioxidizing agents such as tocopherol), thickening agents, wetting and dispersing agents, buffers, humectants, such as lactic acid and glycolic acid copolymers, emulsifying agents, fillers, emollients and surface active agents (such as sorbitane fatty acid esters).

A preferred dimethicone composition is formulated by combining by weight 1 to 50% of a triacylglycerol, wherein the acyl portions are identical or different and represent a caprylyl radical or a capryl radical, 0.8 to 40% of solubilized lanolin alcohol, 1 to 50% of squalene and 0.001 to 25% of dimethicone.

A second preferred dimethicone composition is formulated by combining by weight 1 to 16% of a triacylglycerol, wherein the acyl portions are identical or different and represent a caprylyl radical or a capryl radical, 0.8 to 12.8% of lanolin alcohol solubilized in mineral oil (80% by weight of lanolin in 20% by weight of mineral oil), 1.0 to 16% of squalene, 0.01 to 25% of dimethicone and a buffer.

The following examples will serve to fur(er illustrate the present invention without, at the same time, however, constituting any limitation thereof.

### EXAMPLE 1

### Preparation of Dimethicone Oil

An oil composition containing dimethicone was prepared as follows using the recipe set forth in Table 1.

TABLE 1
Dimethicone Composition Ingredients

| INGREDIENTS | % WEIGHT |
|---|---|
| Dimethicone | 10.0 |
| Heavy mineral oil (Ramol 350) | 79.4 |
| Isopropyl myristate (emulsifying agent) | 10.0 |
| Lanolin alcohol (Amerchol L-101) | 0.5 |
| Sorbic acid | 0.1 |
| Total | 100.0 |

Isopropyl myristate (INOLEX CHEMICAL CO) was heated to 54 to 60° C. in a stainless steel container. Sorbic acid (PENTA MANUFACTURING COMPANY) was added and mixed until completely dissolved. AMERCHOL L-101 (CHARLES TENNANT COMPANY CANADA LTD) was added with mixing, the temperature adjusted to 50-55° C., and mixing continued until the preparation was uniform. Dimethicone (SILICONE SF 96-350, GENERAL ELECTRIC COMPANY) and heavy mineral oil (CANADA COLORS AND CHEMICALS) were added with mixing and mixing continued for at least 15 minutes or until the preparation was uniform in appearance. The dimethicone composition was transferred to a sterile, screw-cap bottle and stored at room temperature until use.

### EXAMPLE 2

### Preparation of Dimethicone Cream

A cream composition containing dimethicone was prepared as follows using the recipe set forth in Table 2.

TABLE 2
Dimethicone Composition Ingredients

| INGREDIENTS | % WEIGHT |
|---|---|
| Dimethicone | 5.00 |
| Glycerin | 8.00 |
| Citric acid anhydrous | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B 1 | 0.50 |
| Purified water | 71.29 |
| Total | 100.00 |

Oil Phase (Phase A):

Atmul 84 (VAN WATERS & ROGERS), Amerchol L-101, cholesterol (CRODA CANADA LTD, Canada), Emulgade 1000 (HENKEL), Eumulgin B1 (HENKEL), dimethicone (Silicone SF 96-350, GENERAL ELECTRIC COMPANY, Canada) were mixed at 72 to 78° C. in a heated kettle.

Aqueous Phase (Phase B):

Purified water and glycerin (CANADA COLORS AND CHEMICALS) were added into a reactor, mixed at 25 RPM, and heated. Citric acid (DEBRO, Canada) and edetate disodium (VAN WATER & ROGERS) were added, the temperature brought to 72 to 78° C. and the ingredients mixed until uniform. Phase A, at 72 to 78° C., was added to phase B, at 72 to 78° C., homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41° C. with mixing at 24 RPM for 20 to 30 minutes. The mixture was then cooled to 33 to 35° C., with mixing at 20 RPM for 20 to 30 minutes. The mixture was then cooled to 24 to 28°C., with mixing at 20 RPM for 20 to 30 minutes. The dimethicone composition was transferred to a sterile, screw-cap jar and stored at room temperature until use.

### EXAMPLE 3

### Preparation of Dimethicone Cream Containing Caprylic/Capric Acid Triglyceride

A dimethicone cream composition containing caprylic/capric acid triglyceride was prepared as follows using the recipe set forth in Table 3 below.

TABLE 3
Dimethicone Composition Ingredients

| INGREDIENTS | % WEIGHT |
|---|---|
| Dimethicone | 5.00 |
| Caprylic/capric acid triglyceride | 5.00 |
| Glycerin | 8.00 |
| Citric acid (anhydrous) | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B 1 | 0.50 |
| Purified water | 66.29 |
| Total | 100.00 |

Oil Phase (Phase A):

Atmul 84, Amerchol L-101, cholesterol, Emulgade 1000, Eumulgin B1, caprylic/capric acid triglyceride (Croda Canada Ltd, Canada) and dimethicone are mixed at 72 to 78° C. in a heated kettle.

Aqueous Phase (Phase B):

Purified water and glycerin were added into a reactor, mixed at 25 RPM, and heated. Citric acid and edetate were added, the temperature brought to 72 to 78° C. and the ingredients mixed until uniform. Phase A, at 72 to78° C., was added to phase B, at 72 to 78° C., homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41° C. with mixing at 24 RPM for 20 to 30 minutes. The mixture was then cooled to 33 to 35° C., with mixing at 20 RPM for 20 to 30 minutes. The mixture was then cooled to 24 to 28° C., with continued mixing at 20 RPM for 20 to 30 minutes. The dimethicone composition was transferred to a sterile, screw-cap jar and stored at room temperature until use.

### EXAMPLE 4

### Preparation of Dimethicone Cream Containing a Protease Inhibitor

A dimethicone cream composition containing a protease inhibitor was prepared as follows using the recipe set forth in Table 4 below.

TABLE 4
Dimethicone Composition Ingredients

| INGREDIENTS | % WEIGHT |
|---|---|
| Dimethicone | 5.00 |
| Protease inhibitor | 1.00 |
| Glycerin | 8.00 |
| Citric acid (anhydrous). | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B1 | 0.50 |
| Purified water | 70.29 |
| Total | 100.00 |

Oil Phase (Phase A):

Atmul 84, Amerchol L-101, cholesterol, Emulgade 1000, Eumulgin B1, and dimethicone were mixed at 72 to 78° C. in a heated kettle.

Aqueous Phase (Phase B):

Purified water and glycerin were added into a reactor, mixed at 25 RPM, and heated. Citric acid and edetate disodium were added, the temperature brought to 72 to 78° C. and the ingredients mixed until uniform. Phase A, at 72 to 78° C., was added to phase B, at 72 to. 78° C., and homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41° C. with mixing at 24 RPM for 20 to 30 minutes. The mixture was then cooled to 33 to 35° C., with mixing at 20 RPM for 20 to 30 minutes. Protease inhibitor was added and the mixture cooled to 24 to 28° C., with mixing at 20 RPM for 20 to 30 minutes. The dimethicone composition was transferred to a sterile, screw-cap jar and stored at room temperature until use.

### EXAMPLE 5

### Preparation of Dimethicone Composition Containing Caprylic/Capric Acid Triglyceride and a Protease Inhibitor

A dimethicone composition containing caprylic/capric acid triglyceride and a protease inhibitor was prepared as follows using the recipe set forth in Table 5 below.

TABLE 5
Dimethicone Composition Ingredients

| INGREDIENTS | % WEIGHT |
|---|---|
| Dimethicone | 5.00 |
| Caprylic/capric triglyceride | 5.00 |
| Protease inhibitor | 1.00 |
| Glycerin | 8.00 |
| Citric acid anh. | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B 1 | 0.50 |
| Purified water | 65.29 |
| Total | 100.00 |

Oil Phase (Phase A):

Atmul 84, Amerchol L-101, cholesterol, Emulgade 1000, Eumulgin B 1, caprylic/capric acid triglyceride and dimethicone were mixed at 72 to 78° C. in a heated kettle.

Aqueous Phase (Phase B):

Purified water and glycerin were added into a reactor, mixed at 25 RPM, and heated. Citric acid and edetate disodium were added, the temperature was brought to 72 to 78° C. and the ingredients were mixed until uniform. Phase A, at 72 to 78° C., was added to phase B, at 72 to 78° C., homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41° C. with mixing at 24 RPM for 20 to 30 minutes. The mixture was then cooled to 33 to 35° C., with mixing at 20 RPM for 20 to 30 minutes. Protease inhibitor was added and the mixture cooled to 24 to 28° C., with mixing at 20 RPM for 20 to 30 minutes. The dimethicone composition was transferred to a sterile, screw-cap jar and stored at room temperature until use.

### EXAMPLE 6

### Preparation of Dimethicone Composition Containing Triacylglycerols, Squalene, and a Protease Inhibitor

A dimethicone composition containing triacylglycerols, squalene and a protease inhibitor was prepared as follows using the recipe set forth in Table 6 below.

TABLE 6
Dimethicone Composition Ingredients

| INGREDIENTS | % WEIGHT |
|---|---|
| Dimethicone | 5.00 |
| Caprylic/capric triglyceride | 5.00 |
| Squalene | 5.00 |
| Protease inhibitor | 1.00 |
| Glycerin | 8.00 |
| Citric acid anh. | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B 1 | 0.50 |
| Purified water | 60.29 |
| Total | 100.00 |

Oil Phase (Phase A):

Atmul 84, Amerchol L-101, cholesterol, Emulgade 1000, Eumulgin B1, capyrlic/capric acid triglyceride, squalene (SUPRAENE, Robeco) and dimethicone were mixed at 72 to 78° C. in a heated kettle.

Aqueous Phase (Phase B):

Purified water and glycerin were added into a reactor, mixed at 25 RPM, and heated.Citric acid and edetate disodium were added, the temperature brought to 72 to 78° C. and the ingredients mixed until uniform. Phase A, at 72 to 78° C., was added to phase B, at 72 to 78° C., homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41° C. with mixing at 24 RPM for 20 to 30 minutes. The mixture was then cooled to 33 to 35° C., with mixing at 20 RPM for 20 to 30 minutes. Protease inhibitor was added and the mixture cooled to 24 to 28° C., with mixing at 20 RPM for 20 to 30 minutes. The dimethicone composition was transferred to a sterile, screw-cap jar and stored at room temperature until use.

### EXAMPLE 7

### Preparation of Dimethicone Cream Composition Containing Caprylic/Capric Acid Triglyceride and Squalene

A dimethicone composition containing caprylic/capric acid triglyceride and squalene was prepared as follows using the recipe set forth in Table 7 below.

TABLE 7
Dimethicone Composition Ingredients

| INGREDIENTS | % WEIGHT |
|---|---|
| Dimethicone | 5.00 |
| Caprylic/capric triglyceride | 5.00 |
| Squalene (SUPRAEME) | 5.00 |
| Glycerin | 8.00 |
| Citric acid anh. | 0.06 |
| Edetate disodium | 0.05 |
| Atmul 84 | 4.00 |
| Lanolin oil (Amerchol L-101) | 3.00 |
| Cholesterol | 0.10 |
| Emulgade 1000 | 8.00 |
| Eumulgin B1 | 0.50 |
| Purified water | 61.29 |
| Total | 100.00 |

Oil Phase (Phase A):

Atmul 84, Amerchol L-101, cholesterol, Emulgade 1000NI Eumulgin B1, capyrlic/capric acid triglyceride, squalene and dimethicone were mixed at 72 to 78° C. in a heated kettle.

Aqueous Phase (Phase B):

Purified water and glycerin were added into a reactor, mixed at 25 RPM, and heated. Citric acid and edetate disodium were added, the temperature brought to 72 to 78° C. and the ingredients mixed until uniform. Phase A, at 72 to 78° C., was added to phase B, at 72 to 78° C., homogenized at 1000 RPM for 5 to 10 minutes with mixing at 25 RPM. After homogenization, the mixture was cooled to 48 to 52° C. with mixing at 28 RPM for 20 to 30 minutes. The mixture was then cooled to 39 to 41°° C. with mixing at 24 RPM for 20 to 30 minutes. The mixture was then cooled to 33 to 35° C., with mixing at 20 RPM for 20 to 30 minutes. The mixture is cooled to 24 to 28° C., with mixing at 20 RPM for 20 to 30 minutes. The dimethicone composition was transferred to a sterile, screw-cap jar and stored at room temperature until use.

### EXAMPLE 8

### Treatment of Inflammation with Dimethicone Cream Composition

Patients with eczema (inflammation) were treated with the dimethicone composition of Example 2. A thin film of the dimethicone composition was topically applied directly to the eczema several times a day for several days. The patients reported a decrease in the inflammation of the eczema. No adverse side effects of the dimethicone composition were reported.

A patient with a burned lower lip was treated with the dimethicone composition of Example 2. The dimethicone composition was topically applied directly to the burned lip three times a day for two weeks. The patient reported a decrease in inflammation and healing of the lip. No adverse side effects of the dimethicone composition were reported.

A patient with a cut on one leg was treated with the dimethicone composition of Example 2. The dimethicone composition was topically applied directly to the cut twice a day for several days. The patient reported a decrease in inflammation and healing of the cut. No adverse side effects of the dimethicone composition were reported.

### EXAMPLE 9

### Treatment of Inflammation with Dimethicone Cream Composition Containing Caprylic/Capric Acid Triglyceride

Patients with contact dermatitis were treated with the dimethicone composition of Example 3. A thin layer of the dimethicone composition was topically applied directly to the rash several times a day for several days. The patients reported resolution of the inflammation. No adverse side effects of the dimethicone composition were reported.

A patient with actinic keratosis lesions was treated with the dimethicone composition of Example 3 twice a day for several weeks. The patient reported a decrease in the inflammation of the lesion and drying, flaking-off and healing of the lesions. No adverse side effects of the dimethicone composition were reported.

Patients with bed sores were treated with the dimethicone composition of Example 3. A layer of the dimethicone composition was applied directly to the bed sore twice a day for a week. The patients reported a marked decrease in the inflammation of the bed sores and an improvement in healing of the bed sores. No adverse side effects of the dimethicone composition were reported.

### EXAMPLE 10

### Effect of Dimethicone on Elastase Activity

Although PMNs play an important role in killing infectious agents, they also contribute to the symptoms of inflammation by their release of proteolytic enzymes such as elastase.

To determine the effect of dimethicone on elastase activity, porcine pancreatic elastase (0.45 μg) in 900 μl of Tris-HCl buffer, pH 8.0, containing 0.5 M NaCl and 100 μg of BSA was incubated at 30° C. in the presence of 0.2, 0.5, 0.7, 1, 1.6 and 2.0% dimethicone. After 15 minutes, 2 mM of the synthetic substrate N-Suc-Ala-Ala-Val-Ala-pNA was added, the incubation was continued for 15 minutes and the reaction was stopped with 100 μl of glacial acetic acid. Reaction product was measured spectrophotometrically at 410 nm.

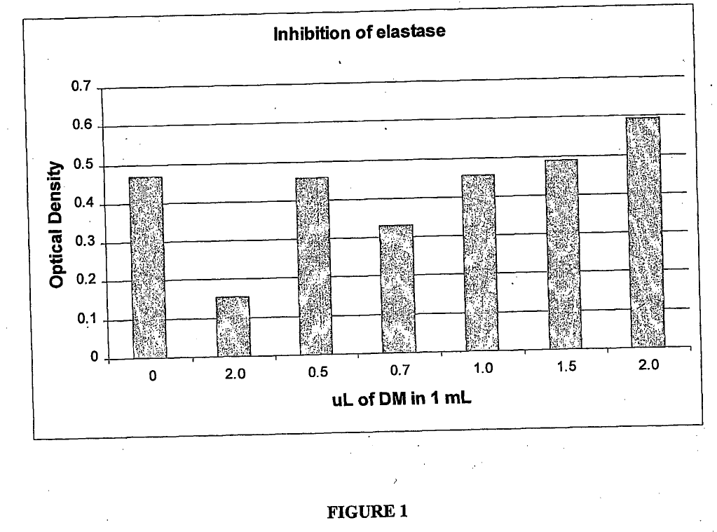

As shown in FIG. 1, 0.2 % of dimethicone was sufficient to significantly inhibit elastase activity. That increasing amounts of dimethicone did not equally inhibit elastase activity may result from the low solubility of dimethicone in aqueous media. That is, as dimethicone is an oil, at high concentrations it may form micelles and is no longer available to interact with the elastase enzyme.

### EXAMPLE 11

### Effect of Dimethicone on PMN and Macrophage Phagocytosis

PMNs are the first line of defense against invading pathogens and play a significant role during inflammation. In response to chemotatic factors, PMNs leave the circulation and invade the inflammed area where they recognize, phagocytize and kill the pathogens. Therefore, the effect of dimethicone on PMN phagocytosis was determined.

Blood was obtained from healthy human volunteers and PMNs were obtained by gradient centrifugation at 600 g for 30 minutes with Lympholyte-poly (Cedarlane, Homby, Canada), followed by hypotonic lysis of contaminating erythrocytes. Cells were suspended in RPMI (Gibco, Burlington, Canada) supplemented with 10% fetal bovine serum (PBS). Final preparations consisted of greater than 95% PMNs as determined by Wright Giemsa staining having greater than 97% viability as determined by trypan blue exclusion.

Blood was obtained from healthy human volunteers and peripheral blood mononuclear leukocytes (hereinafter “PBMLs”) were obtained by gradient centrifugation with Lympholyte-poly (Cedarlane, Homby, Canada) at 600 g for 30 minutes at room temperature. The PBML fraction was collected and washed 3× with 10 ml of phosphate buffered saline (PBS) by centriugation. The cells were suspended in 5 ml of RPMI (Gibco) supplemented with 10% FBS (Hyclone). Viability was greater than 97% as determined by trypan blue exclusion.

To assess the effect of dimethicone on phagocytosis, PMNs and PBMLs (2×10⁶/ml) were incubated at 37° C. in 5% CO₂, 95% humidity in the presence of 0, 2.5 and 5% dimethicone. After 24 hours, viability was determined by trypan blue exclusion and the cells were washed 3× in PBS containing 2 mM glucose, 1 mM MgCl₂ and 1 mM CaCl₂. The cell concentration was adjusted to 1×10⁶ cells/ml and the cells were incubated with fluoresbrite carboxylate microspheres (1/10 dilution). After 30 minutes, the cells were washed, fixed in 2% paraformaldehyde and analyzed for microshpere ingestion using an XL flow Cytometer (Coulter). Data are expressed as percent (%) of phagocytosis.

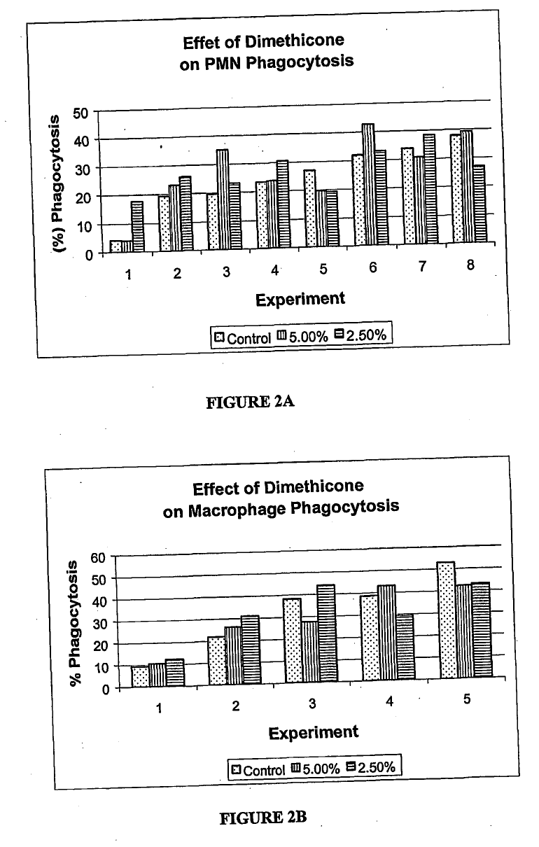

As shown in FIG. 2A, dimethicone stimulated PMN phagocytosis in six and inhibited PMN phagocytosis in two of the experiments. These data show that dimethicone modulates PMN phagocytosis and that the effect observed is dependent on the activated-state of the immune system of the donor. As shown in FIG. 2B, dimethicone had no effect on macrophage phagocytosis.

### EXAMPLE 12

### Effect of Dimethicone on PBML Proliferation

PBMLs were isolated as described in Example 11. The cells (2×10⁵ cells/ml) were incubated in RPMI containing 10% FBS in the wells of 96-well microtiter plates with 0, 10⁻⁵, 10⁻⁴, 10⁻³, 10⁻², 10^{-1,} 1 and 10× PHA (GIBCO, BURLINGTON, CANADA) at 37° C. hours in the presence of 0 or 2.5% dimethicone (100% dimethicone). After 54 hours of incubation, 1 μCi of +8³H]-thymidine was added to each well and the incubation was continued for 18 hours. Plates were harvested on Titertek filters and the radioactivity in each well was determined in a β-counter. Results are expressed in log CPM of incorporated [³H]-thymidine.

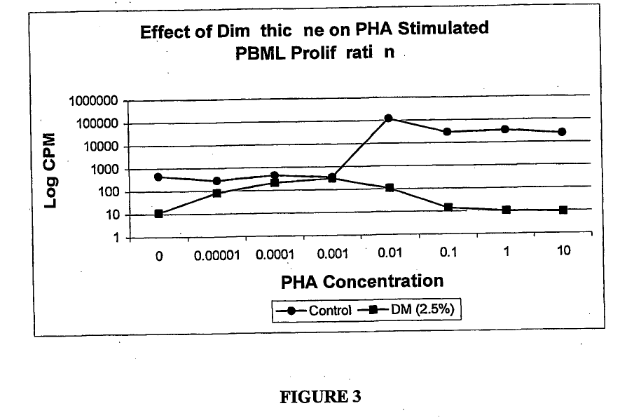

As shown in FIG. 3, dimethicone inhibited PHA-stimulated mitogenic proliferation of PBMLs.

### EXAMPLE 13

### Effects of Dimethicone on Dermal Sensitization in Mice

Delayed type hypersensitivity (DTH) reactions in the skin represent an increasing cause of inflammatory dermatoses typically exemplified by allergic contact dermatitis (eczema). Skin sensitization in mice has been used as an animal model of atopic dermatitis. CD1 mice (20 g) were sensitized to oxazolone (OXA) under ketamine:xylazine anesthesia (0.1 ml/10 g, i.p.) by applying 100 μl of 5% solution of OXA (Sigma Chemicals, St-Louis) in acetone onto 2 cm² area of the abdomen. Three days later, ear thickness was measured with a caliper, at the distal one third of the ear, and 50 μl of 5% OXA in acetone was applied on both faces of the right ear (challenge); the left ear served as control. Mice were distributed in 6 groups (10 animals per group) and treated with hydrocortisone (HC) and dimethicone (DM) according to the following table.

| Group | Treatment |
|---|---|
| A | Control: PEG/EtOH solution |
| B | Hydrocortisone 1% (w/v) in PEG/EtOH |
| C | DM (0.1%, v/v) in PEG/EtOH |
| D | DM (1.0%, v/v) in PEG/EtOH |
| E | DM (5.0%, v/v) in PEG/EtOH |
| F | DM (10.0%, v/v) in PEG/EtOH |

Treatment consisted of applying, twice daily, 25 μl of drug solution on both faces of the right ear. Ear thickness was measured every day and appearance of erythema was noted.

On days 10 and 17, mice were re-challenged by applying the OXA solution on the right ear, followed by the same treatment but only once daily for four days. Mice were sacrificed on day 21.

The mean increase of ear thickness (MIET) was determined by the formula below. Statistical analysis was performed using the Student's t test.

$$MIET = \frac{\begin{matrix}\text{Mean thickness after challenge} - \\ \text{Mean before challenge}\end{matrix}}{\text{Number of mice treated}}$$

Results:

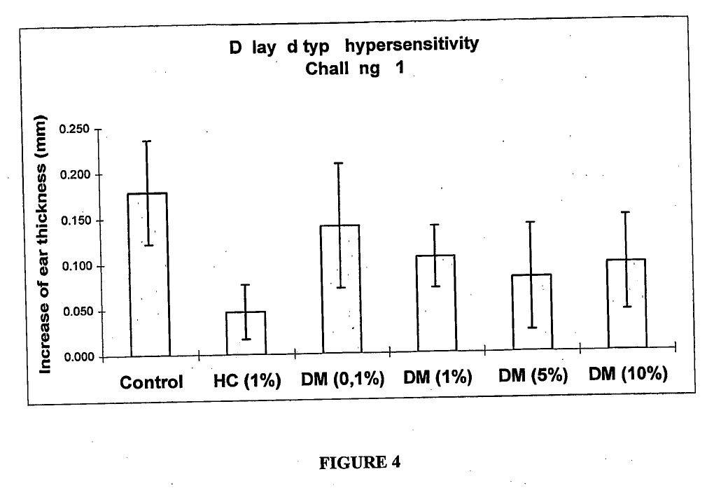

As shown in FIG. 4, control animals had an MIET of 0.179±0.05 mm (n=10). The ears showed acute inflammation as seen by erythema and swelling from 24 hours post challenge through the second challenge. Twice daily application of 1% hydrocortisone (positive control) suppressed by 74% (p<0.00001) the MIET. Twice daily application of varying concentrations of dimethicone also significantly reduced MIET in a dose-dependent fashion. For acute inflammation, a concentration of 0.1% dimethicone had no significant effect. However, at concentrations of 1, 5 and 10%, significant reduction of inflammation (up to 55% inhibition) were observed (p<0.003, 0.0014 and 0.0034 respectively). Both treatment (hydrocortisone and dimethicone) also reduced erythema of the ear.

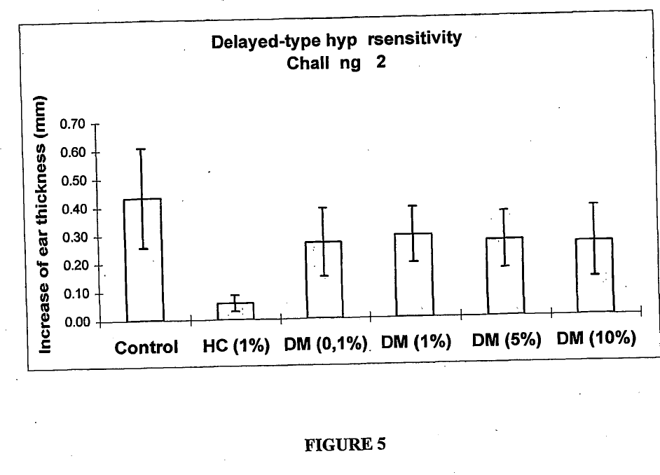

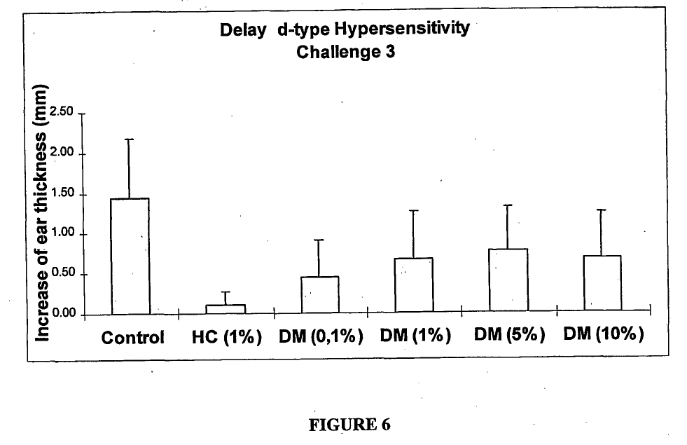

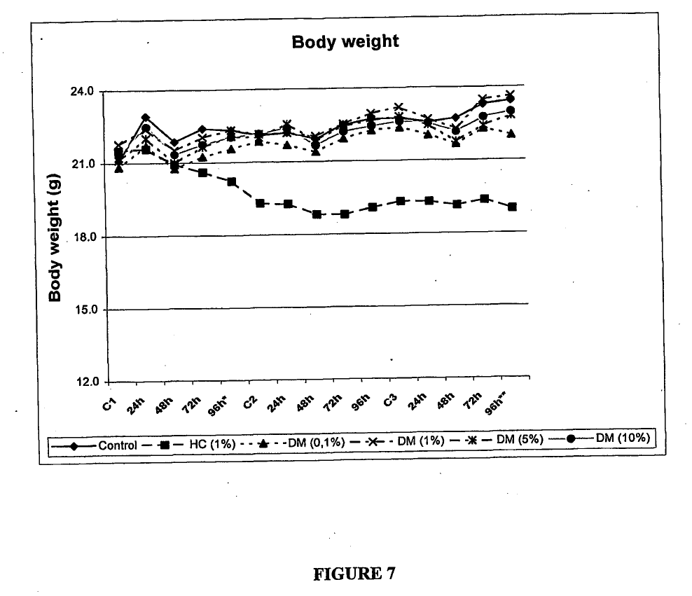

To achieve chronic inflammation (swelling, redness and crust formation), mice were re-challenged at day 10 and 17. On day 14 and 21 respectively, the inflamed ear was 3 to 7 times thicker than the non-challenged ear. As shown in FIG. 5 and 6, all doses of dimethicone inhibit significantly (up to 69%) the MIET. Topical application of dimethicone solution reduced redness, crust formation and ear swelling. Inhibition of inflammation by dimethicone was not as strong as hydrocortisone. However, hydrocortisone exerts a systemic effect as the mice treated with hydrocortisone showed a decrease of body weight (see FIG. 7), probably due to immunosuppression.

All patents, patent applications and scientific articles mentioned herein are hereby incorporated by reference.

Modifications and variations of the present method will be obvious to those skilled in the art from the foregoing detailed description. Such modifications and variations are intended to come within the scope of the appended claims.

1-14. (canceled)

15. A method for treating inflammation of the skin or mucous membrane wherein said method comprises topical administration to a patient in need of such treatment, of an anti-inflammatory amount of dimethicone in the absence of any other anti-inflammatory agent.

16. The method, according to claim 15, wherein the inflammation is selected from the group consisting of acne; eczema; gingivitis; psoriasis; pruritus; decubitus ulcer; allergic dermatitis: seborrheic dermatitis; mummular dermatitis; actinic keratosis; actinic lentigos; ichthyoses; ichtyposiformis; Darier maladay; palmoplantary keratodermies; leucoplasies;, leucoplasiformis; lichen; blisters; collagen maladies; ultraviolet light damage; rosacea; melasma; comedons; polymorphs; conglobata; infection; sinusitis; dyshidrosis tonsillitis; buccal ulceration; gastrointestinal diseases; inflammatory bowel diseases; urogenital diseases; fungal, yeast, bacterial and viral dermatides; and wounds.

17-21. (canceled)

22. The method, according to claim 15, wherein said dimethicone is administered by a route selected from the group consisting of liquids, oils, emulsions, lotions, gels, creams, pastes, ointments, foams, powders, aerosols, inhalants, patches and suppositories

23. A composition formulated for topical administration comprising dimethicone, in the absence of any other anti-inflammatory agent, and a pharmaceutically acceptable carrier.

24-27. (canceled)

28. The composition according to claim 23, which is selected from the group consisting of liquids, oils, emulsions, lotions, gels, creams, pastes, ointments, foams, powders, aerosols, inhalants, patches and suppositories.
